(19) United States
(12) Patent Application Publication
(10) Pub. No.: US 2013/0204143 A1
(43) Pub. Date: Aug. 8, 2013

(54) PULSE WAVE MEASUREMENT APPARATUS AND PROGRAM

(71) Applicant: Seiko Epson Corporation, Tokyo (JP)

(72) Inventor: Atsushi NARUSAWA, Chino-shi (JP)

(73) Assignee: SEIKO EPSON CORPORATION, Tokyo (JP)

(21) Appl. No.: 13/735,492

(22) Filed: Jan. 7, 2013

(30) Foreign Application Priority Data
Feb. 2, 2012 (JP) 2012-020766
Feb. 2, 2012 (JP) 2012-020767
Oct. 1, 2012 (JP) 2012-219093

(51) Int. Cl. A61B 5/024; A61B 5/00

(52) U.S. Cl. CPC A61B 5/02427; A61B 5/7225; A61B 5/7278; A61B 5/7214
USPC 600/479

(57) ABSTRACT

A pulse wave measurement apparatus includes a light emitting device that applies light to a measurement position at which a pulse wave is measured, and a plurality of light receiving devices that output measurement signals based on the amount of received light which is applied from the light emitting device and is reflected from the measurement position. The control unit of the pulse wave measurement apparatus performs the independent component analysis on the measurement signals output from the plurality of light receiving devices, and calculates the weighting factors of each component when each of the measurement signals is divided into a plurality of components. The control unit calculates the dispersion of the calculated weighting factors for each component, and specifies a component in which the calculated dispersion is the smallest. The control unit generates pulse wave information indicative of the pulse wave based on the specified component.

### BACKGROUND

1. Technical Field

The present invention relates to a technology which measures the pulse wave of a living body.

2. Related Art

As a method of detecting a pulse wave of a living body, in particular, a human body, a method of measuring a pulse wave by performing photoelectric conversion (a photoelectric method) has been used. In this method, light of a wave length which is easily absorbed by blood is emitted from a light emitting device such as a light emitting diode. After the light passes through a living body or enters the living body, light which is dispersed due to tissue in the living body is received by a light receiving device, such as a photodiode or a phototransistor. Thereafter, a pulse wave is detected by converting the received light into an electrical signal. Although arteries are repeatedly dilated and constricted at the same period as that of a heartbeat, the degree of absorption of light which enters a living body is higher in a case in which the artery is dilated compared to a case in which the artery is constricted. Therefore, the intensity of light which is received by the light receiving device is changed according to pulsation. That is, the light absorption amount emitted from the light emitting device increases or decreases at a period which is the same as the period at which a blood vessel is dilated and constricted, and the intensity of reflected light is changed according to the increase and decrease. The pulse wave is measured based on the change.

However, when the pulse wave of a living body is measured, there are cases in which noise (hereinafter, referred to as “body motion noise”) is generated in the results of the measurement due to the movement of the living body (hereinafter, referred to as “body motion”). The body motion noise is generated because the pressurization or decompression state of the measurement position and the positional relationship between the light emitting device/the light receiving device and the living body are changed according to the body motion and affect the direction or amount of received light. As a technology which is used to reduce body motion noise, a pulse wave sensor which has a configuration in order to avoid a congestion state is disclosed in JP-A-2002-224064.

Blood vessels which are detection targets are present in the dermis of the skin of the living body. From a surface, capillary vessels and subpapillary vascular plexuses are formed, subcutaneous vascular plexuses are formed further below, and there is a tendency for blood vessels in deep portions to be thick. When a pulse wave is measured using the photoelectric method, the sensitivity of body motion noise differs between cases in which thick blood vessels are present under a sensor and cases in which thick blood vessels are not present under a sensor. When measurement is performed at a position at which thick blood vessels are present under a sensor, the influence of body motion noise increases. In a pressure pulse wave sensor disclosed in JP-A-2002-224064, a congestion state can be avoided but the influence of body motion noise is greatly received according to the position of a sensor.

### SUMMARY

An advantage of some aspects of the invention is to reduce the influence of noise which is generated due to the operation of a measurement position in a pulse wave measurement apparatus.

An aspect of the invention is directed to a pulse wave measurement apparatus including: a plurality of light receiving units that output measurement signals each indicative of a received amount of light which is irradiated to a measurement position where pulse waves are measured, and passes through or is reflected from the measurement position; an independent component analysis unit that performs an independent component analysis on the measurement signals which are output from the plurality of light receiving units, divides each of the measurement signals into a plurality of components, and calculates weighting factors of each of the plurality of components; a dispersion calculation unit that calculates a value indicative of a degree of dispersion of values of the weighting factors which are calculated by the independent component analysis unit for each of the plurality of components; a component specification unit that specifies a component of the plurality of components whose value indicative of the degree of dispersion calculated by the dispersion calculation unit satisfies a predetermined condition; and a pulse wave information generation unit that generates pulse wave information indicative of the pulse waves using the component specified by the component specification unit. According to this aspect, it is possible to reduce the influence of noise which is generated due to the operation of the measurement position in the pulse wave measurement apparatus.

The pulse wave measurement apparatus according to the aspect of the invention may be configured such that the pulse wave information generation unit extracts the component specified by the component specification unit by performing an operation using the weighting factors calculated by the independent component analysis unit on the measurement signals which are output from the plurality of light receiving units, and generates the pulse wave information indicative of the pulse waves based on the extracted component. According to this configuration, it is possible to reduce the influence of noise which is generated due to the operation of the measurement position in the pulse wave measurement apparatus.

The pulse wave measurement apparatus according to the aspect of the invention may be configured such that the component specification unit specifies a component whose value indicative of the degree of dispersion calculated by the dispersion calculation unit is smallest. According to this configuration, it is possible to reduce the influence of noise which is generated due to the operation of the measurement position in the pulse wave measurement apparatus.

The pulse wave measurement apparatus according to the aspect of the invention may be configured such that the component specification unit specifies a component whose value indicative of the degree of dispersion calculated by the dispersion calculation unit is within a predetermined range. According to this configuration, it is possible to reduce the influence of noise which is generated due to the operation of the measurement position in the pulse wave measurement apparatus.

The pulse wave measurement apparatus according to the aspect of the invention may be configured such that, when the plurality of components are specified by the component specification unit, the pulse wave information generation unit generates the plurality of specified components, and generates the pulse wave information based on the sum of the plurality of generated components. According to this configuration, it is possible to reduce the influence of noise which is generated due to the operation of the measurement position in the pulse wave measurement apparatus.

The pulse wave measurement apparatus according to the aspect of the invention may be configured such that the pulse wave measurement apparatus further includes a measurement signal specification unit that specifies a measurement signal whose value of the weighting factor of the component specified by the component specification unit satisfies the predetermined condition from among the measurement signals output from the plurality of light receiving units and the pulse wave information generation unit generates the pulse wave information indicative of the pulse waves based on the measurement signal specified by the measurement signal specification unit. According to this configuration, it is possible to reduce the influence of noise which is generated due to the operation of the measurement position in the pulse wave measurement apparatus.

The pulse wave measurement apparatus according to the aspect of the invention may be configured such that the component specification unit specifies a component whose value indicative of the degree of dispersion calculated by the dispersion calculation unit is greatest. According to this configuration, it is possible to reduce the influence of noise which is generated due to the operation of the measurement position in the pulse wave measurement apparatus.

The pulse wave measurement apparatus according to the aspect of the invention may be configured such that the measurement signal specification unit specifies a measurement signal whose value of the weighting factor of the component specified by the component specification unit is smallest. According to this aspect, it is possible to reduce the influence of noise which is generated due to the operation of the measurement position in the pulse wave measurement apparatus.

The pulse wave measurement apparatus according to the aspect of the invention may be configured such that the pulse wave information generation unit removes the component specified by the component specification unit from the measurement signal specified by the measurement signal specification unit, and generates the pulse wave information based on the measurement signal from which the component is removed. According to this configuration, it is possible to reduce the influence of noise which is generated due to the operation of the measurement position in the pulse wave measurement apparatus.

Another aspect of the invention is directed to a program causing a computer to perform: receiving measurement signals from a plurality of light receiving units that output the measurement signals each indicative of a received amount of light which is irradiated to a measurement position where pulse waves are measured, and passes through or is reflected from the measurement positions; performing an independent component analysis on the measurement signals which are output from the plurality of light receiving units, dividing each of the measurement signals into a plurality of components, and calculating weighting factors of each of the plurality of components; calculating a dispersion value indicative of a degree of dispersion of values of the weighting factors which are calculated in the independent component analysis for each of the plurality of components; specifying a component of the plurality of components whose value indicative of the degree of dispersion calculated in the calculating of the dispersion value satisfies a predetermined condition; and generating pulse wave information indicative of the pulse waves using the component specified in the specifying of the component. According to this aspect, it is possible to reduce the influence of noise which is generated due to the operation of the measurement position in the pulse wave measurement apparatus.

### BRIEF DESCRIPTION OF THE DRAWINGS

The invention will be described with reference to the accompanying drawings, wherein like numbers reference like elements.

FIG. 1 is a view illustrating an appearance of a pulse wave measurement apparatus.

FIG. 2 is a block diagram illustrating an example of the configuration of the pulse wave measurement apparatus.

FIGS. 3A and 3B are views illustrating the positional relationship between a light emitting device and light receiving devices.

FIGS. 4A to 4D are views each illustrating an example of a measurement signal.

FIG. 5 is a block diagram illustrating the function of a control unit.

FIG. 6 is an operational flowchart illustrating the operation of the pulse wave measurement apparatus.

FIGS. 7A and 7B are views illustrating the sensing results of the pulse wave measurement apparatus.

FIG. 8 is a circuit diagram illustrating the light receiving device and a signal processing unit.

FIG. 9 is a block diagram illustrating the function of a control unit.

FIG. 10 is an operational flowchart illustrating the operation of the pulse wave measurement apparatus.

FIGS. 11A and 11B are views illustrating the sensing results of the pulse wave measurement apparatus.

FIG. 12 is a circuit diagram illustrating the light receiving device and the signal processing unit.

FIG. 13 is a view illustrating the positional relationship between a light emitting device and the light receiving devices.

### DESCRIPTION OF EXEMPLARY EMBODIMENTS

### First Embodiment

### Configuration

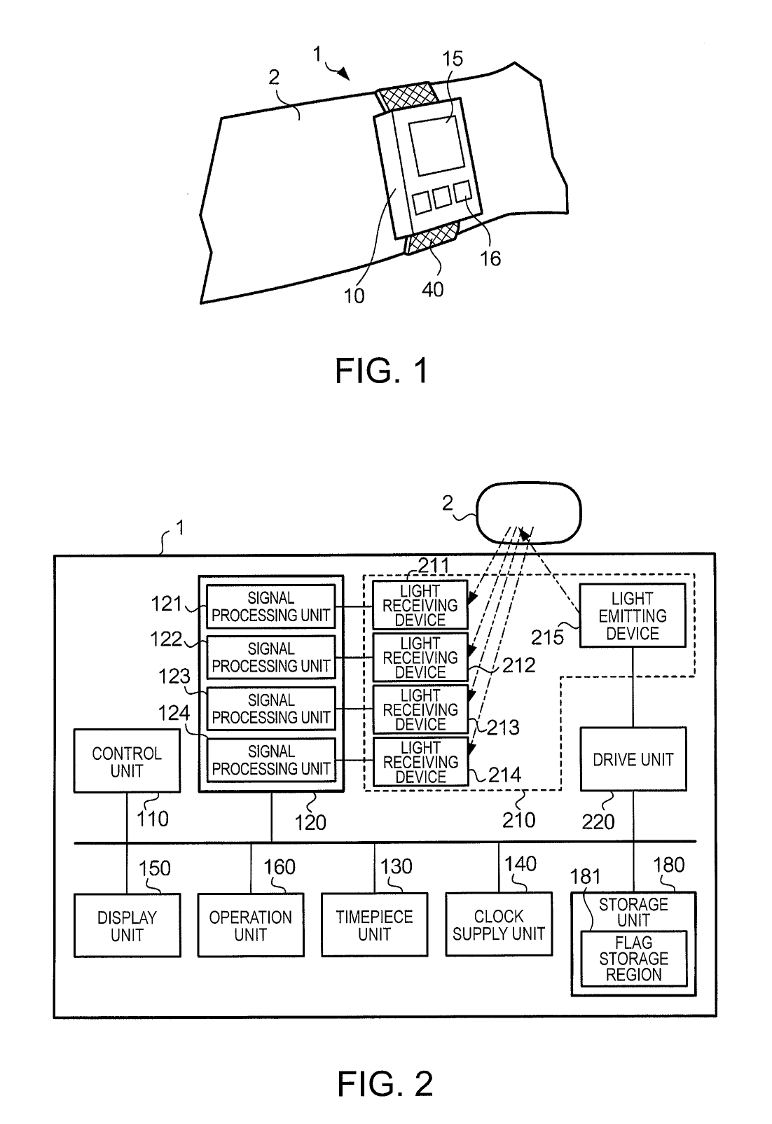

FIG. 1 is a view illustrating the appearance of a pulse wave measurement apparatus 1 according to the first embodiment. As shown in FIG. 1, the pulse wave measurement apparatus 1 is fastened to an arm 2 of a user, and measures the pulse wave of the corresponding position. The pulse wave measurement apparatus 1 includes a main body 10 provided with a display 15 and operational switches 16 which are used to operate the pulse wave measurement apparatus 1, and a band 40 which is used to fasten the main body 10 of the apparatus to the arm 2.

FIG. 2 is a block diagram illustrating the configuration of the pulse wave measurement apparatus 1. In the drawing, a light emitting and receiving unit 210 includes a light emitting device 215, for example, a Light Emitting Diode (LED) which irradiates light having a wavelength of green light, and light receiving devices 211, 212, 213, and 214, such as photodiodes which receive green light. The light emitting device 215 is an example of a light emitting unit according to the invention. Light irradiated by the light emitting device 215 reaches the inside of the arm 2, and is reflected in the blood vessels. Reflection occurs in the positions of a plurality of blood vessels, and thus as a whole dispersion-like reflection occurs. This reflected light is received by the light receiving devices 211, 212, 213, and 214, and the light receiving devices 211, 212, 213, and 214 output signals based on the amount of received light. The light receiving devices 211, 212, 213, and 214 are examples of a plurality of light receiving units according to the invention. A control signal which is used to control the light emission intensity and the light emission timing of the light emitting device 215 is provided to a drive unit 220 from an analog control circuit (not shown), and the drive unit 220 supplies current, the amount of which is based on the amplitude of the control signal, to the light emitting device 215 of the light emitting and receiving unit 210.

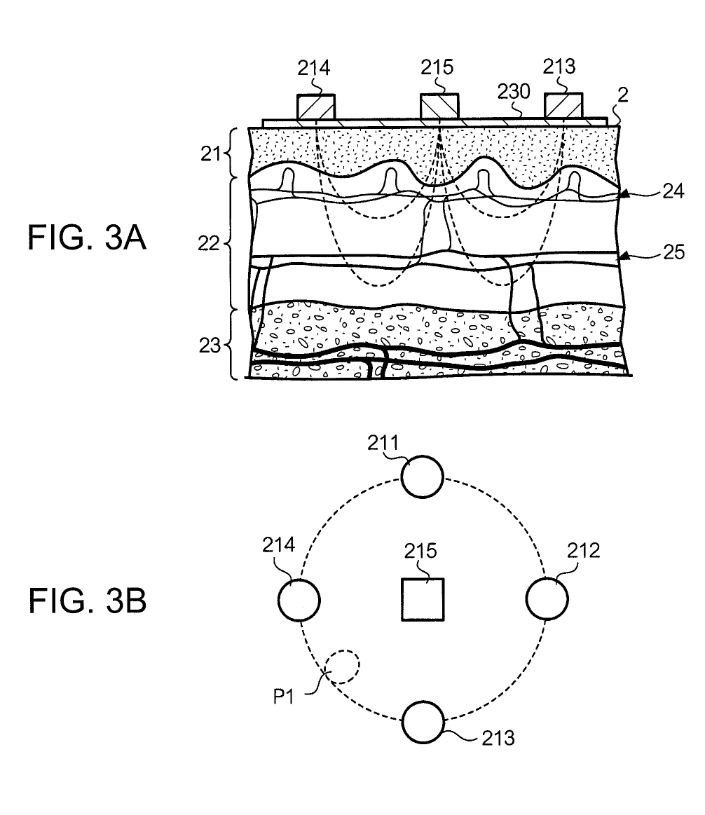

FIG. 3A is a view illustrating the positional relationship between the light emitting device and the light receiving devices, and FIG. 3B is a view illustrating the positional relationship between the light emitting device 215 and the light receiving devices 211, 212, 213, and 214 when viewed from the inside of the arm 2 which is a measurement position (a region in which blood vessels are located). In FIG. 3A, a cross section of the arm 2 is shown. An arm of a human body includes an epidermis 21, a dermis 22 which is under the epidermis 21, and subcutaneous tissue 23 which is under the dermis 22. Capillary vessels 24 are present in the shallow portions of the dermis 22. Fibrillation veins (a general term for an arteriole and a venule) 25 are present in the deep portions of the dermis 22. In FIG. 3A, a transmission board 230, such as a glass plate, which transmits light is provided on a portion of the pulse wave measurement apparatus 1 which comes into contact with the arm 2. The light emitting device 215 and the light receiving devices 211, 212, 213, and 214 are provided on the upper surface of the transmission board 230. Light which is emitted from the light emitting device 215 passes through the epidermis 21 and the dermis 22, and then is received by the respective light receiving devices 211, 212, 213, and 214 through reflection.

As shown in FIG. 3B, the light receiving devices 211, 212, 213, and 214 are arranged at approximately equal intervals on the circumference having the light emitting device 215 as a center thereof. The light emitting device 215 is fixed on the plane of the transmission board 230 such that the vertical direction thereof is substantially an optical axis direction, and the light receiving devices 211, 212, 213, and 214 are fixed on the plane of the transmission board 230 such that the light receiving surfaces substantially face the vertical direction. Light emitted from the light emitting device 215 passes through the transmission board 230 and is applied to a measurement position, thus light which is reflected from the measurement position is received by the light receiving devices 211, 212, 213, and 214 through the transmission board 230. Each of the light receiving devices 211, 212, 213, and 214 outputs a measurement signal of the amount of current based on the amount of received light. In the description below, for convenience of description, the measurement signal which is output from the light receiving device 211 is set to “measurement signal G₁”. In the same manner, description will be made while the measurement signal which is output from the light receiving device 212 is set to “measurement signal G₂”, the measurement signal which is output from the light receiving device 213 is set to “measurement signal G₃”, and the measurement signal which is output from the light receiving device 214 is set to “measurement signal G₄”.

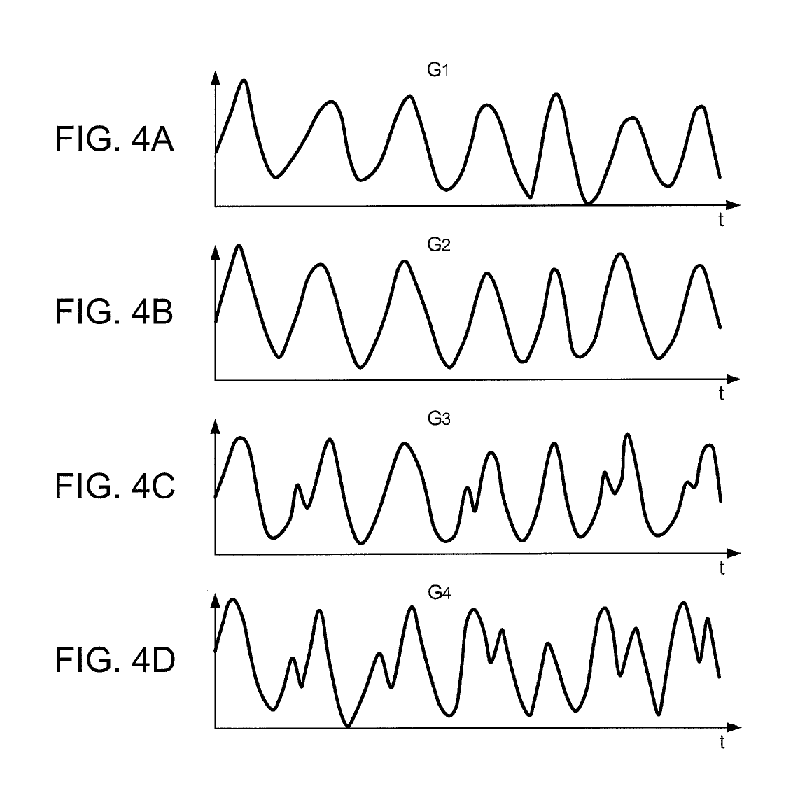

FIGS. 4A to 4D are views schematically illustrating examples of waveforms shown by measurement signals G₁, G₂, G₃, and G₄. FIG. 4A illustrates the measurement signal G₁, FIG. 4B illustrates the measurement signal G₂, FIG. 4C illustrates the measurement signal G₃, and FIG. 4D illustrates the measurement signal G₄. When there are deep portions of the dermis 22 or when there are thick blood vessels in the subcutaneous tissue 23, there are cases in which the direction or amount of light is greatly changed according to body motion due to thick blood vessels. More specifically, for example, in FIG. 3B, when a thick blood vessel is positioned at a position P1, the measurement signals (that is, the measurement signal G₃ and the measurement signal G₄) which are output from the light receiving device 213 and the light receiving device 214 are greatly affected by body motion noise. On the contrary, the measurement signals (that is, the measurement signal G₁ and the measurement signal G₂) which are output from the light receiving device 211 and the light receiving device 212 are measurement signals which are not affected by the body motion noise to such a degree.

In FIG. 2, a control unit 110 includes a Central Processing Unit (CPU) and memories (a Read Only Memory (ROM) and a Random Access Memory (RAM)), and controls each unit which is connected to the control unit 110 in such a way that the CPU executes a control program stored in the ROM. More specifically, the control unit 110 performs a process of generating pulse wave information (described later) in response to the measurement signals G₁, G₂, G₃, and G₄ which are output from the respective light receiving devices 211, 212, 213, and 214.

A signal processing unit 120 includes signal processing units 121, 122, 123, and 124. The signal processing unit 121 includes an amplifier (not shown) which acquires the measurement signal G₁ output from the light receiving device 211 and amplifies the measurement signal G₁, and an A/D conversion circuit (not shown) which quantizes the amplified measurement signal G₁ using a predetermined sampling frequency. The signal processing unit 122 includes an amplifier (not shown) which acquires the measurement signal G₂ output from the light receiving device 212 and amplifies the measurement signal G₂, and an A/D conversion circuit (not shown) which quantizes the amplified measurement signal G₂ using a predetermined sampling frequency. The signal processing unit 123 includes an amplifier (not shown) which acquires the measurement signal G₃ output from the light receiving device 213 and amplifies the measurement signal G₃, and an A/D conversion circuit (not shown) which quantizes the amplified measurement signal G₃ using a predetermined sampling frequency. The signal processing unit 124 includes an amplifier (not shown) which acquires the measurement signal G₄ output from the light receiving device 214 and amplifies the measurement signal G₄, and an A/D conversion circuit (not shown) which quantizes the amplified measurement signal G₄ using a predetermined sampling frequency. A timepiece unit 130 counts the time keeping clock signal of a clock supply unit 140 and times time. The clock supply unit 140 includes an oscillation circuit and a clock division circuit, supplies a reference clock signal to the control unit 110 by the oscillation circuit, and generates a time keeping clock signal for timing and supplies the generated time keeping clock signal to the control unit 110 by the clock division circuit. A display unit 150 includes a display 15, and displays various types of images, such as time information timed by the timepiece unit 130, a menu screen used to measure a pulse wave, and results of measurement, under the control of the control unit 110. An operation unit 160 includes an operational switch 16, and transmits an operation signal acquired through operations performed by the operational switch 16 to the control unit 110. A storage unit 180 includes a flag storage region 181 which stores a flag which is referred to when a pulse wave information generation process which will be described later is performed.

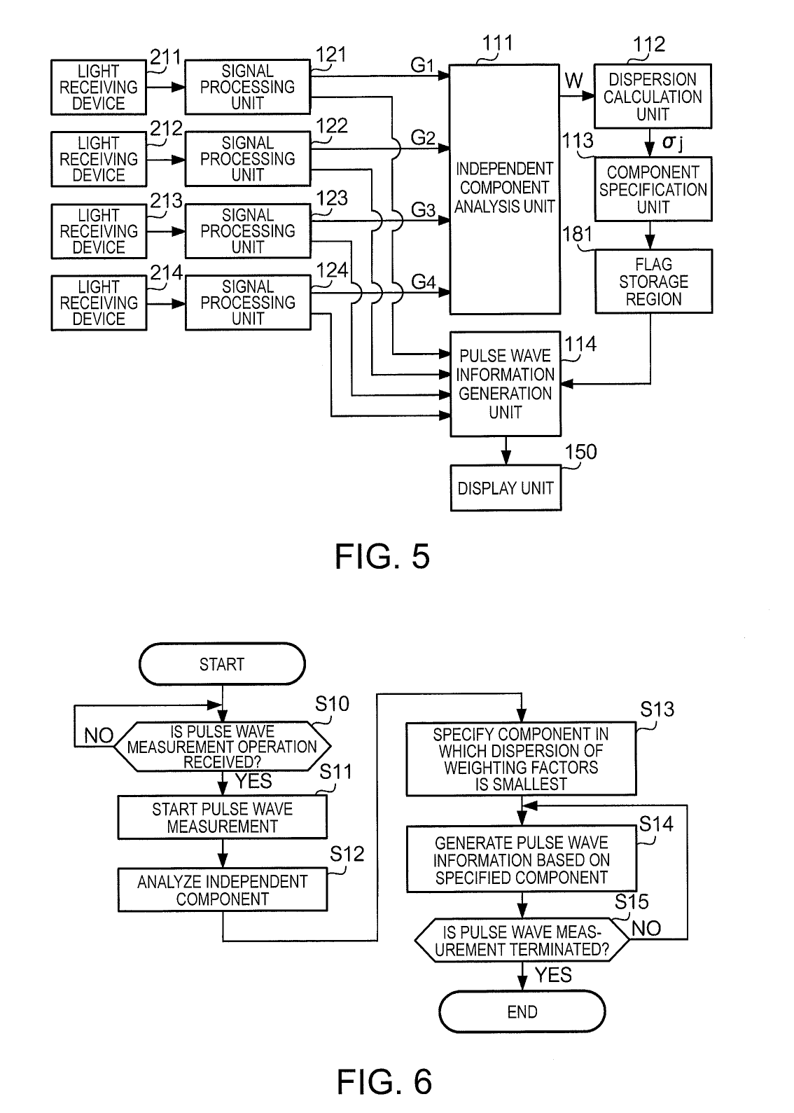

FIG. 5 is a functional block diagram illustrating a functional block (which includes a part of the configuration shown in FIG. 2 other than the control unit 110) used to implement the function of a pulse wave measurement process by the control unit 110. An independent component analysis unit 111, a dispersion calculation unit 112, a component specification unit 113, and a pulse wave information generation unit 114 shown in the drawing are executed in such a way that the control unit 110 reads and executes a computer program stored in the ROM.

The independent component analysis unit 111 performs an independent component analysis (ICA) on a measurement signal Gᵢ (1≦i≦n; n is an integral number which is equal to or greater than 2) which is output from each of a plurality of light receiving devices, and acquires the weighting factor wᵢⱼ of each component in a case in which the measurement signal Gᵢ is divided into a plurality of components Sⱼ (1≦j≦m; where n≧m, m is an integral number which is equal to or greater than 2). The plurality of components indicate a signal which is output from each signal source in a case in which it is assumed that a plurality of signal sources are present in a region in a body which is a measurement target. Here, each of the signal sources generates a signal based on the movement of a blood vessel which is in a measurement target region, and it is possible to specify a signal source which locates at a position where it is difficult to receive the body movement based on analysis using Equations which will be described later.

In this embodiment, the independent component analysis unit 111 performs the independent component analysis on the measurement signals G₁, G₂, G₃, and G₄ which are output from the respective light receiving devices 211, 212, 213, and 214, and acquires the weighting factors w₁₁, w₁₂, w₄₄ of each of the components in a case in which each of the measurement signals G₁, G₂, G₃, G₄ is divided into a plurality of components S₁, S₂, S₃, and S₄. In the first embodiment, as described below, in a case in which it is assumed that an array G is Equation 1, an array S is Equation 2, and an array W is Equation 3, the independent component analysis unit 111 performs the independent component analysis and estimates four independent components S₁, S₂, S₃, and S₄ which cause the arrays G, S, and W to satisfy Equation 4. That is, the independent component analysis unit 111 calculates the weighting factors w₁₁, w₁₂, . . . w₄₄ using any one of a method based on non-Gaussian maximization, a method based on maximum likelihood estimation, a method based on mutual information amount, a method based on uncorrelated nonlinear functions, and a method based on tensors such that the components S₁, S₂, S₃, and S₄ are statistically independent from each other. The independent component analysis performed by the independent component analysis unit 111 uses, for example, a method written by Noboru Murata, “introduction to independent component analysis”, first edition, Tokyo Denki University Press, July 2004”.

$$\begin{matrix}G = ( \begin{matrix}{G}_{1} \\ {G}_{2} \\ {G}_{3} \\ {G}_{4}\end{matrix} ) \\ G = WS , & ( 1 ) \\ S = ( \begin{matrix}{S}_{1} \\ {S}_{2} \\ {S}_{3} \\ {S}_{4}\end{matrix} ) , & ( 2 ) \\ W = ( \begin{matrix}{w}_{11} & {w}_{12} & {w}_{13} & {w}_{14} \\ {w}_{21} & {w}_{22} & {w}_{23} & {w}_{24} \\ {w}_{31} & {w}_{32} & {w}_{33} & {w}_{34} \\ {w}_{41} & {w}_{42} & {w}_{43} & {w}_{44}\end{matrix} ) & ( 3 )\end{matrix}$$

that is,

$$( \begin{matrix}{G}_{1} \\ {G}_{2} \\ {G}_{3} \\ {G}_{4}\end{matrix} ) = ( \begin{matrix}{w}_{11} & {w}_{12} & {w}_{13} & {w}_{14} \\ {w}_{21} & {w}_{22} & {w}_{23} & {w}_{24} \\ {w}_{31} & {w}_{32} & {w}_{33} & {w}_{34} \\ {w}_{41} & {w}_{42} & {w}_{43} & {w}_{44}\end{matrix} ) ( \begin{matrix}{S}_{1} \\ {S}_{2} \\ {S}_{3} \\ {S}_{4}\end{matrix} ) = ( \begin{matrix}{w}_{11} {S}_{1} + {w}_{12} {S}_{2} + {w}_{13} {S}_{3} + {w}_{14} {S}_{4} \\ {w}_{21} {S}_{1} + {w}_{22} {S}_{2} + {w}_{23} {S}_{3} + {w}_{24} {S}_{4} \\ {w}_{31} {S}_{1} + {w}_{32} {S}_{2} + {w}_{33} {S}_{3} + {w}_{34} {S}_{4} \\ {w}_{41} {S}_{1} + {w}_{42} {S}_{2} + {w}_{43} {S}_{3} + {w}_{44} {S}_{4}\end{matrix} ) \qquad ( 4 )$$

A dispersion calculation unit 112 calculates the dispersion σⱼ (the degree of dispersion) of the values of the weighting factors wᵢⱼ, acquired by the independent component analysis unit 111, for each component Sⱼ. In the first embodiment, the dispersion σⱼ indicates the degree of the dispersion of values using a numerical value. For example, the dispersion when the weighting factors include 2, 3.5, 1, and 5 may be less than the dispersion when the weighting factors include 1, 13, −2, and 50. Here, the dispersion σⱼ is defined as a standard deviation. Therefore, the dispersion σⱼ shows that the greater the degree of dispersion is the greater the value of the dispersion is and that the lower the value is the lower the degree of dispersion is. The dispersion calculation unit 112 calculates the dispersion σⱼ of the weighting factor wᵢⱼ corresponding to the component Sⱼ (that is, the weighting factor wᵢⱼ included in the j-th column of the array W). That is, the dispersion calculation unit 112 calculates the dispersion of the weighting factor wᵢⱼ included in the array W for each column. More specifically, in the first embodiment, dispersion σ₁ indicates the degree of dispersion of the weighting factors w₁₁, w₂₁, w₃₁, and w₄₁, and dispersion σ₂ indicates the degree of dispersion of the weighting factors w₁₂, w₂₂, w₃₂, and w₄₂. Dispersion σ₃ indicates the degree of dispersion of the weighting factors w₁₃, w₂₃, w₃₃, and w₄₃, and dispersion σ₄ indicates the degree of dispersion of the weighting factors w₁₄, w₂₄, w₃₄, and w₄₄. The embodiment of the calculation of the degree of dispersion of the weighting factors performed by the dispersion calculation unit 112 is not limited to the calculation of the standard deviation for each component, and any calculation in which the degree of dispersion of weighting factors is calculated may be used.

A component specification unit 113 specifies one or more components which satisfy a condition in which the dispersion σⱼ calculated by the dispersion calculation unit 112 is previously determined. In the first embodiment, the component specification unit 113 calculates the calculated dispersion σⱼ for each of the components S₁, S₂, S₃, and S₄, and specifies a component in which the dispersion σⱼ of the weighting factors is the smallest. The component specification unit 113 stores information (flag) indicative of the specified component in a flag storage region 181. The flag is referred to by the pulse wave information generation unit 114.

A pulse wave information generation unit 114 generates pulse wave information indicative of a pulse wave based on the measurement signals output from the signal processing unit 120. In the first embodiment, the pulse wave information generation unit 114 extracts a component (hereinafter, referred to as “specified component”) which is specified by the component specification unit 113 by performing an operation on the measurement signals G₁, G₂, G₃, and G₄ output from the signal processing unit 120 using the weighting factor wᵢⱼ. Since the measurement signals G₁, G₂, G₃, and G₄ and the components S₁, S₂, S₃, and S₄ satisfy the above-described Equation 4, the component Sⱼ is calculated using the following Equation 5. The pulse wave information generation unit 114 extracts the specified component using the following Equation 5.

$$S = ( \begin{matrix}{S}_{1} \\ {S}_{2} \\ {S}_{3} \\ {S}_{4}\end{matrix} ) = {W}^{- 1} G \qquad ( 5 )$$

As described above, there are cases in which the component of body motion noise (hereinafter, referred to as “noise component”) is mixed with the measurement signal Gᵢ according to the position of the light receiving device due to the influence of the thick blood vessels in the deep portions of the dermis 22 or the subcutaneous tissue 23. The magnitude of the influence of the noise component varies according to the position of the light receiving device, and the magnitude of the influence greatly varies even when the position of the light receiving device is slightly deviated. More specifically, in the example shown in FIG. 3B, in a case in which a thick blood vessel which is a noise source is located at a position P1, the value of the weighting factor of the noise component which is extracted from the measurement signal G₃ and the measurement signal G₄ is great but the value of the weighting factor of the noise component which is extracted from the measurement signal G₁ and the measurement signal G₂ is small. As described above, the weighting factor of the noise component extracted from the measurement signal Gᵢ differs in each of the measurement signals Gᵢ in which the positions of the light receiving devices are different from each other. Therefore, with respect to the component Sⱼ, it is considered that the dispersion of the weighting factors is great in a component which has a large number of noise components than a component which has a small number of noise components. Based on the above-described reason, among the components S₁, S₂, S₃, and S₄ estimated by performing the independent component analysis, it is possible to assume that the component Sⱼ which has the least dispersion σⱼ of the weighting factors is a component with which the smallest body motion noise is mixed. The embodiment has a configuration in which the influence of noise is suppressed by using the component Sⱼ which has the least dispersion σⱼ of the weighting factors as a signal used to measure a pulse wave.

The pulse wave information generation unit 114 generates pulse wave information indicative of a pulse wave based on the calculated specified component. In the first embodiment, the pulse wave information generation unit 114 assumes that a time interval between the peaks of the wave form of the calculated specified component is a pulse period and that the appearance frequency of peaks of the wave form of the measurement signal during a predetermined time (1 minute or the like) is a pulse rate, and outputs information (pulse wave information) indicative of the pulse period and the pulse rate on the display unit 150. In addition, the pulse wave information generation unit 114 may, for example, chronologically store the information indicative of the pulse period and the pulse rate in the storage unit 180.

### Operation Example

FIG. 6 is a view illustrating the flow of the operation of the pulse wave measurement apparatus 1. Hereinafter, an example of the operation of the pulse wave measurement apparatus 1 according to the first embodiment will be described with reference to FIG. 6. A measurement target person first performs an operation of starting the measurement of a pulse wave by the operational switch 16. When the control unit 110 receives an operation of measuring a pulse wave (step S10; YES), the process proceeds to step S11. That is, the control unit 110 applies light to a measurement position, receives light reflected from the measurement position, and starts to output measurement signals based on the amount of received light in step S11. The control unit 110 performs A/D conversion on the measurement signals which are output from the respective light receiving devices 211, 212, 213, and 214 by the signal processing unit 120, acquires the chronological measurement signals from the signal processing unit 120, and stores the acquired measurement signals in the RAM.

When the output of the measurement signals starts, the control unit 110 performs the independent component analysis on the measurement signals G₁, G₂, G₃, and G₄, which are output from the respective light receiving devices 211, 212, 213, and 214 and on which the A/D conversion is performed by the signal processing unit 120, in step S12, divides each of the measurement signals G₁, G₂, G₃, and G₄ into the plurality of components S₁, S₂, S₃, and S₄, and acquires weighting factors w₁₁, w₁₂, . . . , and w₄₄ which are related to each of the components.

Subsequently, the control unit 110 calculates the dispersion σᵢ of the acquired weighting factors for each of the components S₁, S₂, S₃, and S₄, specifies a component, in which the dispersion is the smallest, in step S13, and stores information indicative of the specified component (specific character component) in the flag storage region 181.

Once the processes up to step S13 have been completed, the control unit 110 generates pulse wave information based on the component specified in step S13, and displays an image indicative of the pulse wave information on the display unit 150. More specifically, the control unit 110 calculates the specified component using the measurement signals G₁, G₂, G₃, and G₄ which are output from the signal processing unit 120 in step S14, detects the time interval between peaks of the waveform of the calculated specified component as a pulse period and the appearance frequency of the peaks of the waveform of the measurement signal during a predetermined time as a pulse rate, and displays an image indicative of the detected pulse period and pulse rate (pulse wave information) on the display unit 150. Meanwhile, if the control unit 110 does not receive the operation of measuring a pulse wave through the operation unit 160 (step S10; NO), the control unit 110 stands by until the operation is performed.

The control unit 110 repeatedly performs the process in step S14 until an operation of terminating the measurement of the pulse wave is performed through the operation unit 160 (step S15; NO). The control unit 110 terminates the process when the operation of terminating the measurement of the pulse wave is performed through the operation unit 160 (step S15; YES).

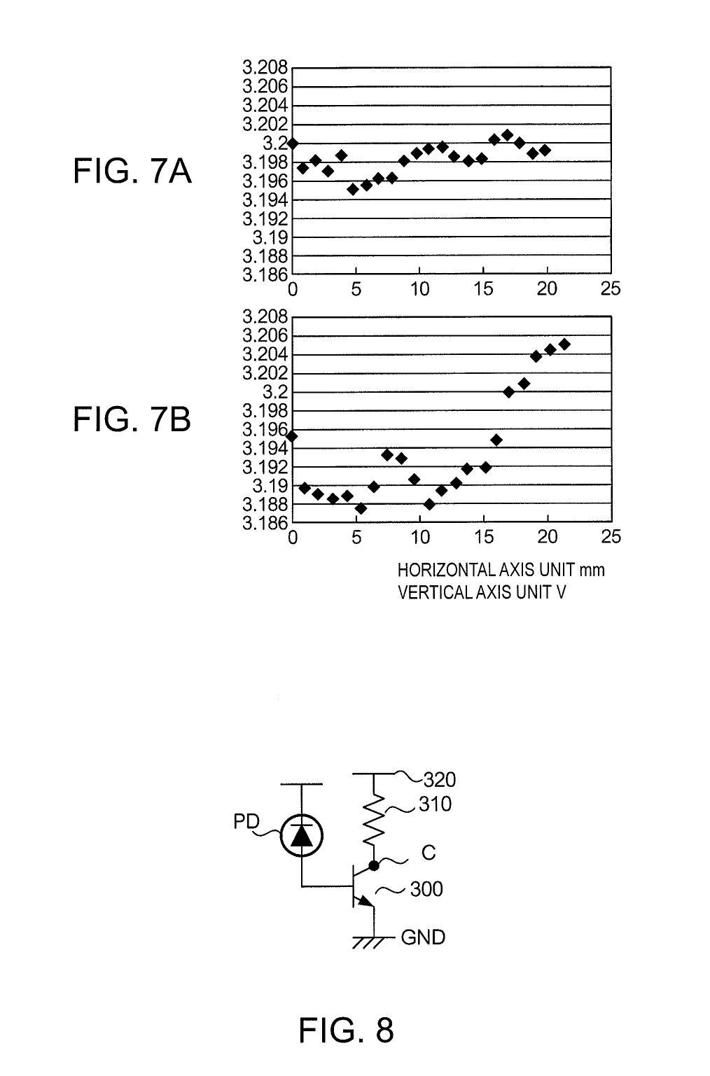

FIG. 7A is a view illustrating an example of pulse wave information which is measured by the pulse wave measurement apparatus 1 (hereinafter, referred to as “sensing results”) and FIG. 7B is a view illustrating an example of the sensing results of a pulse wave measurement apparatus of the related art. In addition, FIG. 8 is an example of a circuit diagram including the light receiving device and the signal processing unit and is a view illustrating an example of a circuit diagram used to perform measurement which produces the sensing results shown in FIGS. 7A and 7B. In FIGS. 7A and 7B, each horizontal axis shows the position of a light receiving device (a distance from a predetermined reference position) (mm), and each vertical axis shows electrical potential (V). The reference position indicates the position of the light receiving device which corresponds to the measurement position of a living body when the measurement starts. As shown in FIG. 8, a light receiving device PD, such as a photodiode, is connected to the base of a transistor 300, a terminal 320 which applies a predetermined voltage is connected to a collector C via a resistor 310, and an emitter is grounded by the ground GND. The light receiving device PD corresponds to each of the light receiving devices 211, 212, 213, and 214 according to the first embodiment, and the transistor 300 corresponds to each of the signal processing units 121, 122, 123, and 124 according to the above-described embodiment. That is, the pulse wave measurement apparatus 1 according to the first embodiment includes four sets of the light receiving device PD and the transistor 300 shown in FIG. 8.

FIG. 7B is a graph illustrating collector electrical potential which is measured by the collector C when it is assumed that the resistance of the resistor 310 is 10 KΩ and a voltage at the terminal 320 is 3.3 V in the circuit diagram shown in FIG. 8. Meanwhile, FIG. 7A is a graph illustrating components which are specified by performing the above-described independent component analysis, the dispersion calculation process, and the component specification process on electrical potentials (that is, the measurement signals G₁, G₂, G₃, and G₄) which are measured by respective four collectors C when it is assumed that the resistance of the resistor 310 is 10 KΩ and the voltage at the terminal 320 is 3.3 V in the circuit diagram shown in FIG. 8.

Due to the difference in density of the blood vessels of a living body and the influence of the distribution of blood vessels having different thicknesses, the level of each of the collector electrical potentials varies according to the measurement position of the living body as shown in FIG. 7B. That is, if the measurement position is deviated, there is a great possibility that measurement is performed for blood of a different tissue as a target thereof. Therefore, as shown in FIG. 7B, even when a slight deviation is generated in the measurement position, the error thereof is great. In contrast, in FIG. 7A, variation in the level of the electrical potential attributable to the change in the measurement position is less. Meanwhile, the sensing results shown in FIGS. 7A and 7B vary according to a circuit multiplier, such as the amount of light of the LED, the sensitivity of the photodiode, or resistance, and values shown in FIGS. 7A and 7B are only for reference.

In the first embodiment, among the components S₁, S₂, S₃, and S₄ which are estimated by performing the independent component analysis, a component, in which the dispersion of the weighting factors is the smallest, is used as the component of a signal used to measure a pulse wave. Since the component in which dispersion of the weighting factors is the smallest is a component in which the influence of noise is the smallest, the measurement of a pulse wave, in which the influence of noise is suppressed, is performed by using such a signal.

### Second Embodiment

Subsequently, a second embodiment according to the invention will be described. The second embodiment and the above-described first embodiment differ in that the processes performed by the control unit 110 are different, and the other processes are the same as those in the above-described first embodiment. Here, description will be made below while focusing on the differences between the second embodiment and the first embodiment. In addition, for the components of the second embodiment which are the same as the components of the first embodiment, the same reference numerals are used as in the first embodiment.

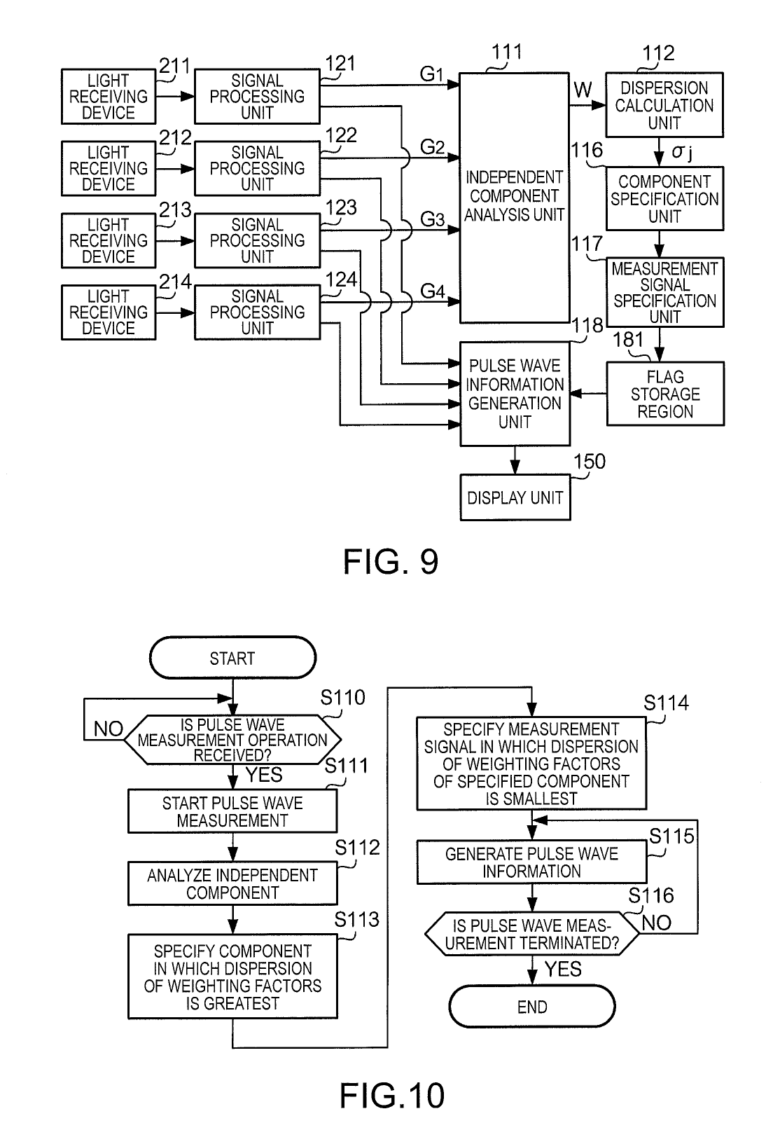

FIG. 9 is a view illustrating the configuration (which includes a part of the configuration shown in FIG. 2 other than a control unit 110) of functional blocks which are used to implement the functions of the pulse wave measurement process performed by the control unit 110 according to the second embodiment. FIG. 9 corresponds to the functional block diagram shown in FIG. 5 in the above-described first embodiment. An independent component analysis unit 111, a dispersion calculation unit 112, a component specification unit 116, a measurement signal specification unit 117, and a pulse wave information generation unit 118 shown in the drawing are implemented in such a way that the control unit 110 reads and executes a computer program stored in a ROM. The independent component analysis unit 111 and the dispersion calculation unit 112 are the same as those in the above-described first embodiment, and the description thereof is omitted here.

The component specification unit 116 specifies one or more components Sⱼ in which dispersion σⱼ calculated by the dispersion calculation unit 112 satisfies a predetermined condition. In the second embodiment, the component specification unit 116 specifies the component Sⱼ in which the calculated dispersion σⱼ is the greatest (that is, j is specified).

As described above, according to the position of the light receiving device, there are cases in which a noise component is mixed due to the influence of the deep portions of the dermis 22 or a thick blood vessel in the subcutaneous tissue 23. The magnitude of the influence of the noise component differs according to the position of the light receiving device, and the magnitude of the influence is greatly changed even when the position of the light receiving device is slightly deviated. More specifically, for example, in the example shown in FIG. 3B, when a thick blood vessel which is a noise source is positioned at the position P1, the weighting factors of the noise components extracted from the measurement signal G₃ and the measurement signal G₄ have the great values, and the weighting factors of the noise components extracted from the measurement signal G₁ and the measurement signal G₂ have the small values. As described above, the weighting factor of the noise component extracted from the measurement signal Gᵢ differs in each measurement signal Gᵢ in which the positions of the light receiving devices are different from each other. In addition, with respect to the component Sⱼ, it is considered that the dispersion of the weighting factors is great in a component which has a large number of noise components than a component which has a small number of noise components. Therefore, it is considered that the component Sⱼ, whose value of the dispersion of the weighting factors σⱼ is large, is a component which includes a large number of noise components because dispersion is great in each of the measurement signals G₁, G₂, G₃, and G₄. In the second embodiment, the component Sⱼ in which the dispersion of the weighting factors σⱼ is great is specified as a component which includes a large number of noise components, a measurement signal Gᵢ in which the specified component is the smallest is selected from among the plurality of measurement signals G₁, G₂, G₃, and G₄, and a pulse wave is measured using the selected measurement signal Gᵢ.

The measurement signal specification unit 117 specifies a measurement signal Gᵢ, whose value of the weighting factor wᵢⱼ (that is, a weighting factor wᵢⱼ included in a j-th column of the array W) of the component Sⱼ specified by the component specification unit 116 is the smallest, from among the measurement signals G₁, G₂, G₃, and G₄. For example, when the component S₁ is specified by the component specification unit 116, the measurement signal specification unit 117 specifies i, the value of which is the smallest, of the weighting factor wᵢ₁ (that is, w₁₁, w₂₁, w₃₁, and w₄₁). When the subscript i is specified, the measurement signal Gᵢ is specified. In addition, for example, when the component S₂ is specified by the component specification unit 116, the measurement signal specification unit 117 specifies i, the value of which is the smallest, of the weighting factor wᵢ₂ (that is, w₁₂, w₂₂, w₃₂, and w₄₂).

Since the measurement signal Gᵢ specified by the measurement signal specification unit 117 is a measurement signal in which the weighting factor wᵢⱼ of the component Sⱼ specified as a component which includes a large number of noise components is the smallest, the measurement signal Gᵢ is a measurement signal in which the influence of noise components is the smallest. As described above, the second embodiment is configured such that a measurement signal in which the influence of noise is the smallest is selected from among the measurement signals output from the plurality of light receiving devices, and a pulse wave is measured using the selected measurement signal. The measurement signal specification unit 117 stores information (a flag) indicative of the specified measurement signal Gᵢ in the flag storage region 181. The flag is referred to by the pulse wave information generation unit 118.

The pulse wave information generation unit 118 generates pulse wave information indicative of a pulse wave based on the measurement signal specified by the measurement signal specification unit 117. In the second embodiment, the pulse wave information generation unit 118 regards a time interval between the peaks of the wave form of the measurement signal specified by the measurement signal specification unit 117 as a pulse period, regards the appearance frequency of peaks of the wave form of the measurement signal during a predetermined time (1 minute) as a pulse rate, and outputs information (pulse wave information) indicative of the pulse period and the pulse rate on the display unit 150. In addition, the pulse wave information generation unit 118 may, for example, chronologically store the information indicative of the pulse period and the pulse rate in the storage unit 180.

### Operation Example

FIG. 10 is a view illustrating the flow of the operation of the pulse wave measurement apparatus 1. Hereinafter, an example of the operation of the pulse wave measurement apparatus 1 according to the second embodiment will be described with reference to FIG. 10. A measurement target person first performs an operation of starting the measurement of a pulse wave by the operational switch 16. When the control unit 110 receives an operation of measuring a pulse wave (step S110; YES), the control unit 110 applies light to a measurement position, receives light reflected from the measurement position, and starts to output measurement signals based on the amount of received light in step S111. The control unit 110 performs A/D conversion on the measurement signals which are output from the respective light receiving devices 211, 212, 213, and 214 by the signal processing unit 120, acquires the chronological measurement signals from the signal processing unit 120, and stores the acquired measurement signals in the RAM.

When the output of the measurement signals starts, the control unit 110 performs the independent component analysis on the measurement signals G₁, G₂, G₃, and G₄, which are output from the respective light receiving devices 211, 212, 213, and 214 and on which the A/D conversion is performed by the signal processing unit 120, in step S112, divides each of the measurement signals G₁, G₂, G₃, and G₄ into the plurality of components S₁, S₂, S₃, and S₄, and acquires weighting factors w₁₁, w₁₂, . . . , and w₄₄ which are related to each of the components.

Subsequently, the control unit 110 calculates the dispersion σⱼ of the acquired weighting factors for each of the components Sᵢ, S₂, S₃, and S₄, and specifies a component Sⱼ, in which the dispersion is the greatest, in step S113. Among the measurement signals G₁, G₂, G₃, and G₄ in step S114, the control unit 110 specifies a measurement signal in which the weighting factor wᵢⱼ of the component Sⱼ which is specified in step S113 is the smallest, and stores information indicative of the specified measurement signal in the flag storage region 181.

Once the processes up to step S114 have been completed, the control unit 110 generates pulse wave information indicative of a pulse wave based on the measurement signal specified in step S114 in step S115. More specifically, the control unit 110 detects, from among the measurement signals G₁, G₂, G₃, and G₄ which are output from the signal processing unit 120, a time interval between peaks of the wave form of a measurement signal indicated by the flag stored in the flag storage region 181 as a pulse period, an appearance frequency of peaks of the waveform of the measurement signal during a predetermined time as a pulse rate, and displays an image indicative of the detected pulse period and the pulse rate on the display unit 150. Meanwhile, if the control unit 110 does not receive the operation of measuring a pulse wave through the operation unit 160 (step S110; NO), the control unit 110 stands by until the operation is performed.

The control unit 110 repeatedly performs the process in step S115 until an operation of terminating the measurement of the pulse wave is performed through the operation unit 160 (step S116; NO). The control unit 110 terminates the process when the operation of terminating the measurement of the pulse wave is performed through the operation unit 160 (step S116; YES).

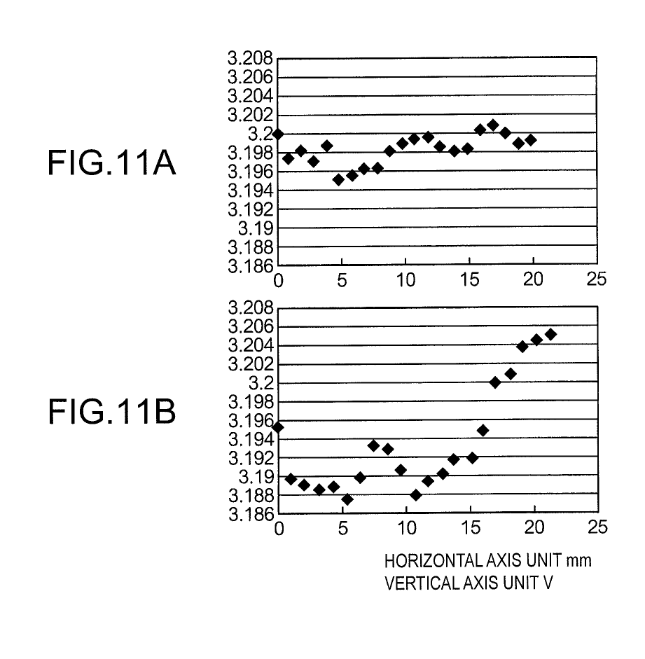

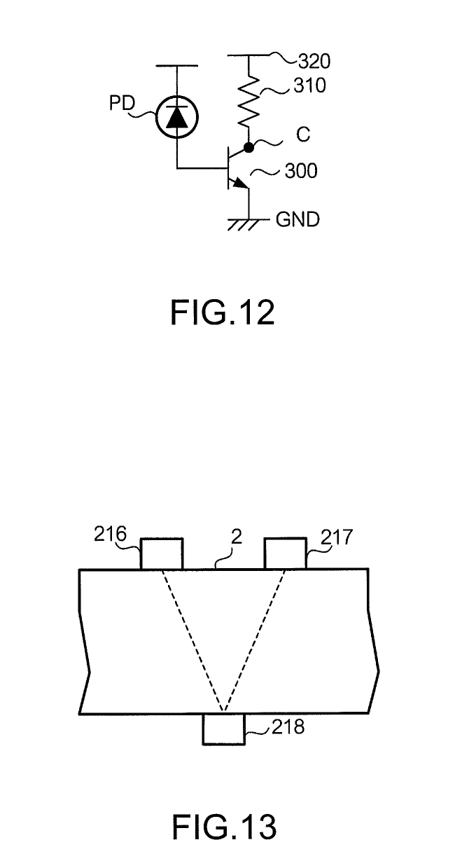

FIG. 11A is a view illustrating an example of sensing results obtained by the pulse wave measurement apparatus 1 and FIG. 11B is a view illustrating an example of the sensing results of a pulse wave measurement apparatus in the related art. In addition, FIG. 12 is an example of a circuit diagram including the light receiving device and the signal processing unit and is a view illustrating a circuit diagram used to perform measurement which produces the sensing results shown in FIGS. 11A and 11B. In FIGS. 11A and 11B, each horizontal axis shows the position of a light receiving device (a distance from a predetermined reference position) (mm), and each vertical axis shows electrical potential (V). The reference position indicates the position of the light receiving device which corresponds to the measurement position of a living body when the sensing results shown in FIGS. 11A and 11B are produced. As shown in FIG. 12, a light receiving device PD, such as a photodiode, is connected to the base of a transistor 300, a terminal 320 which applies a predetermined voltage is connected to a collector C via a resistor 310, and an emitter is grounded by the ground GND. The light receiving device PD corresponds to each of the light receiving devices 211, 212, 213, and 214 according to the second embodiment, and the transistor 300 corresponds to each of the signal processing units 121, 122, 123, and 124 according to the above-described embodiment. That is, the pulse wave measurement apparatus 1 according to the second embodiment includes four sets of the light receiving device PD and the transistor 300 shown in FIG. 12.

FIG. 11B is a graph illustrating collector electrical potential which is measured by the collector C when it is assumed that the resistance value of the resistor 310 is 10 KΩ and a voltage at the terminal 320 is 3.3 V in the circuit shown in FIG. 12. Meanwhile, FIG. 11A is a graph illustrating a measurement signal which is selected by performing the above-described component specification process and the measurement signal specification process on electrical potentials (that is, the measurement signals G₁, G₂, G₃, and G₄) which are measured by respective four collectors C when it is assumed that the resistance value of the resistor 310 is 10 KΩ and the voltage at the terminal 320 is 3.3 V in the circuit shown in FIG. 12.

Due to the difference in density of the blood vessels of a living body and the influence of the distribution of blood vessels having different thicknesses, the level of each of the results of measurement varies according to the measurement position of the living body as shown in FIG. 11B. That is, if the measurement position is deviated, there is a great possibility that measurement is performed for blood of a different tissue as a target thereof. Therefore, as shown in FIG. 11B, even when slight deviation is generated in the measurement position, the error thereof is great. In contrast, in FIG. 11A, variation in the level of the electrical potential attributable to the change in the measurement position is less. Meanwhile, the results of detection shown in FIGS. 11A and 113 vary according to a circuit multiplier, such as the amount of light of the LED, the sensitivity of the photodiode, or resistance, and values shown in FIGS. 11A and 11B are only for reference.

In the second embodiment, among the components S₁, S₂, S₃, and S₄ which are estimated by performing the independent component analysis, a component, in which the dispersion of the weighting factors is the greatest, is specified as a component which includes a large number of noise components, and a measurement signal in which the influence of the specified component is the smallest is used as a signal used to measure the pulse wave. The measurement of a pulse wave, in which the influence of body motion noise is suppressed, is performed using such a measurement signal.

### Modification Example

The invention is not limited to the above-described embodiments, and may be performed in such a way as to be modified as below. In addition, modification examples below may be combined.

(1) In each of the above-described embodiments, the pulse wave measurement apparatus 1 has the configuration shown in FIG. 1. However, the configuration of the pulse wave measurement apparatus 1 is not limited thereto and other configurations may be used. For example, a configuration in which the main body of a device which does not include a light emitting device and light receiving devices are connected to a pulse wave sensor which includes a light emitting device or light receiving devices through a cable may be used. In addition, a configuration in which the main body is connected to the pulse wave sensor using wireless communication may be used. In addition, in the above-described embodiments, the example in which the measurement position on which the pulse wave measurement apparatus 1 is mounted on an arm has been described. However, the measurement position on which the pulse wave measurement apparatus 1 is mounted may be other parts such as the back of a hand or a finger.

(2) In each of the above-described embodiments, the light emitting and receiving unit 210 has the configuration which includes the light emitting device 215 that emits light of a wave length of green light, and the light receiving devices 211, 212, 213, and 214 which receive light of a wave length of green light. However, light which is emitted and received by the light emitting and receiving units is not limited to green light, and light of other wave lengths, such as blue light or infrared light, may be used. In addition, in the above-described embodiments, the light emitting and receiving unit 210 has the configuration which includes the single light emitting device 215 and the plurality of light receiving devices 211, 212, 213, and 214. However, the number of light emitting devices may be plural. For example, a configuration in which the light receiving device corresponds to the light emitting device one to one may be used. In this case, like the above-described first embodiment, the control unit 110 specifies a component in which the dispersion of the weighting factors satisfies a predetermined condition by performing the above-described independent component analysis, the dispersion calculation process, and the component specification process on the measurement signals which are indicative of the results of detection performed by the respective light receiving devices. In addition, as well as in the above-described second embodiment, even when the light receiving device corresponds to the light emitting device one to one, the control unit 110 specifies a measurement signal in which the component in which the dispersion of the weighting factors satisfies the predetermined condition is the smallest by performing the above-described independent component analysis, the dispersion calculation process, and the component specification process on the measurement signals which are indicative of the results of detection performed by the respective light receiving devices.

(3) In each of the above-described embodiments, the pulse wave measurement apparatus 1 has the configuration which includes four light receiving devices 211, 212, 213, and 214. However, the number of light receiving devices is not limited to four and a larger or smaller number of light receiving devices may be used. In addition, in the above-described embodiments, the control unit 110 estimates four components S₁, S₂, S₃, and S₄ by performing the independent component analysis on each of four measurement signals G₁, G₂, G₃, and G₄ which are output from the respective four light receiving devices 211, 212, 213, and 214. However, the number of measurement signals and the number of estimated components are not limited to four and a larger or smaller number of measurement signals and a larger or smaller number of estimated components may be used. In brief, the control unit 110 may estimate a plurality of components (independent components) by performing the independent component analysis on a plurality of measurement signals. However, it is necessary that the number of measurement signals n and the number of estimated components m satisfy a relationship of n m. The condition n≧m is a condition which is necessary to estimate components using the independent component analysis.

(4) In the above-described first embodiment, the control unit 110 estimates a component in which the dispersion of the values of the weighting factors is the smallest from among the plurality of components which are estimated by performing the independent component analysis, and generates pulse wave information using the specified component. The component specification embodiment is not limited thereto. For example, the control unit 110 may specify two components, that is, a component in which the dispersion of the weighting factors is the smallest and a component in which the dispersion of the weighting factors is the second smallest. When the plurality of components are specified, the control unit 110 may calculate the sum of the plurality of specified components (or the sum of weightings), and may generate the pulse wave information based on the results of calculation.

In addition, as another example of the component specification embodiment according to the above-described first embodiment, for example, the control unit 110 may specify one or more components in which the dispersion σᵢ of the values of the weighting factors is smaller than a predetermined threshold (the degree of dispersion is within a predetermined range). In brief, the control unit 110 may specify a component in which the dispersion of the values of the weighting factors satisfies a predetermined condition (dispersion is small). In addition, when a plurality of components are specified, the control unit 110 may calculate the sum of the plurality of specified components (or the sum of weightings), and may generate the pulse wave information based on the results of calculation.

(5) In the above-described first embodiment, the control unit 110 performs the component specification process (the processes in steps S12 to S13 in FIG. 6) at a timing in which the operation of measuring a pulse wave is performed by a measurement target person. However the timing by which the component specification process is performed is not limited to the above timing. For example, the component specification process may be performed at a timing in which the power of the pulse wave measurement apparatus 1 is turned on. In addition, for example, the component specification process may be performed at a timing in which an operation of performing initial setting is performed by the measurement target person. In brief, the control unit 110 may perform the component specification process at any of the timings, and may measure the pulse wave using the specified component when the pulse wave measurement process is performed.

(6) The above-described first embodiment has the configuration in which the control unit 110 specifies a component, in which the dispersion of the weighting factors satisfies a predetermined condition, from among the components estimated by performing the independent component analysis on the plurality of measurement signals, and measures the pulse wave using the specified component, thereby reducing errors generated due to body motion noise which varies according to a measurement position. In addition to this, a configuration in which the control unit 110 performs a process of removing body motion noise (for example, body motion noise generated due to blood congestion) which does not vary due to a measurement position may be used. More specifically, for example, the control unit 110 may determine whether or not blood congestion exists by measuring the change in the volume of blood vessels at the measurement position, and, when it is determined to be the blood congestion, may perform a filtering process of removing a noise component generated due to the blood congestion on the specified component.

(7) In addition, as another example, for example, a configuration in which an acceleration sensor is provided in the pulse wave measurement apparatus 1 is used, and an operation of the measurement target person may be detected by the acceleration sensor, and the control unit 110 may determine whether or not there is blood congestion based on the results of detection. In this case, for example, when the control unit 110 may detect the direction of the measurement position by the acceleration sensor, and may determine whether or not the posture of the measurement position is easy to be congested due to the influence of weight. When it is determined that the posture of the measurement position is easy to be congested, the control unit 110 may perform the filtering process of removing a noise component generated due to the blood congestion on the specified component based on a predetermined algorithm.

(8) In the above-described second embodiment, the control unit 110 specifies a component in which the dispersion of the values of the weighting factors is the greatest as a component which includes a large amount of noise from among the plurality of components estimated by performing the independent component analysis. The component specification embodiment is not limited thereto. For example, the control unit 110 may specify two components, that is, a component in which the dispersion of the weighting factors is the greatest and a component in which the dispersion of the weighting factors is the second greatest. In addition, as another example of the component specification embodiment, for example, the control unit 110 may specify one or more components in which the dispersion σᵢ of the values of the weighting factors is greater than a predetermined threshold. In brief, the control unit 110 may specify a component in which the dispersion of the values of the weighting factors satisfies a predetermined condition (dispersion is great).

When a plurality of components is specified as a component which includes a large amount of noise, the control unit 110 may calculate, for example, the total sum of the weighting factors of the specified component for each measurement signal, and may specify a measurement signal in which the result of calculation is the smallest as a measurement signal which is used to generate the pulse wave information. More specifically, for example, when two components, that is, the component S₁ and the component S₂ are specified as components which include a large amount of noise, the control unit 110 may calculate the sum of the weighting factor wᵢ₁ of the component S₁ and the weighting factor wᵢ₂ of the component S₂ (w₁₁+w₁₂+w₂₁+w₂₂+w₃₁+w₃₂, and w₄₁+w₄₂) for each measurement signal Gᵢ, and may specify the measurement signal Gᵢ in which the result of calculation is the smallest. In addition, as another example, for example, when a plurality of components is specified as a component which includes a large amount of noise, the control unit 110 may calculate the product of the weighting factors of the specified component for each measurement signal, and may specify a measurement signal in which the result of calculation is the smallest. In brief, any configuration in which the control unit 110 specifies a measurement signal which satisfies a predetermined condition (it is considered that the influence of noise is small) from among the measurement signals Gᵢ may be used.

(9) In the above-described second embodiment, the control unit 110 specifies a measurement signal at a timing in which the operation of measuring a pulse wave is performed by a measurement target person. However the timing in which the measurement signal specification process is performed is not limited to the above timing. For example, the measurement signal specification process may be performed at a timing in which the power of the pulse wave measurement apparatus 1 is turned on. In addition, for example, the measurement signal specification process may be performed at a timing in which an operation of performing initial setting is performed by the measurement target person. In brief, the control unit 110 may perform the measurement signal specification process at any of the timings, and may measure the pulse wave using the specified measurement signal when the pulse wave measurement process is performed.

(10) The above-described second embodiment has the configuration in which the control unit 110 specifies one or more components as a component which includes a large amount of noise from among components estimated by performing the independent component analysis on the plurality of measurement signals, and measures the pulse wave using a measurement signal in which the weighting factors of the specified component is the smallest, thereby reducing errors generated due to body motion noise which varies according to a measurement position. In addition to this, a configuration in which the control unit 110 performs a process of removing body motion noise (for example, body motion noise generated due to blood congestion) which does not vary due to a measurement position may be used. More specifically, for example, the control unit 110 may determine whether or not blood congestion exists by measuring the change in the volume of blood vessels at the measurement position, and, when it is determined to be the blood congestion, may perform a filtering process of removing a noise component generated due to the blood congestion on the specified measurement signal.

In addition, as another example, for example, a configuration in which an acceleration sensor is provided in the pulse wave measurement apparatus 1 is used, and an operation of the measurement target person may be detected by the acceleration sensor, and the control unit 110 may determine whether or not there is blood congestion based on the results of detection. In this case, for example, when the control unit 110 may detect the direction of the measurement position by the acceleration sensor, and may determine whether or not the posture of the measurement position is easy to be congested due to the influence of weight. When it is determined that the posture of the measurement position is easy to be congested, the control unit 110 may perform the filtering process of removing a noise component generated due to the blood congestion on the specified measurement signal based on a predetermined algorithm.

(11) In the above-described second embodiment, a configuration in which the control unit 110 removes a component Sⱼ which includes a large amount of noise and which is specified in step S113 from the measurement signal specified in step S114, and generates a pulse wave information based on the measurement signal from which the component Sⱼ which includes a large amount of noise is removed may be used. Since the measurement signals G₁, G₂, G₃, and G₄ and the components S₁, S₂, S₃, and S₄ satisfy the above-described Equation 4, the component Sⱼ is calculated using the above-described Equation 5. The pulse wave information generation unit 118 specifies the component Sⱼ which includes a large amount of noise using the above-described Equation 5, and performs the filtering process of removing the specified component Sⱼ from the measurement signal Gᵢ specified in step S14.

(12) FIG. 13 is a view illustrating an example of the positional relationship between the light emitting device of the pulse wave measurement apparatus according to the modification example of the invention and the light receiving devices. The light receiving devices 211, 212, 213, and 214 of the pulse wave measurement apparatus 1 according to the above-described embodiments receive the reflected light of light applied to the measurement position. However, as shown in FIG. 13, it may be configured such that a plurality of light receiving devices 216 and 217 receives light which is applied to the measurement position from a light emitting device 218 and which passes through the measurement position.

(13) The above-described first embodiment may be combined with the second embodiment. That is, a configuration may be used in which the control unit 110 of the pulse wave measurement apparatus 1 generates pulse wave information based on a component which is specified by the component specification unit 113 (refer to FIG. 5) and a measurement signal which is specified by the measurement signal specification unit 117 (refer to FIG. 9).

(14) A program which is executed by the control unit 110 of the pulse wave measurement apparatus 1 may be provided in a state in which the program is stored in a magnetic recording medium such as a magnetic tape or a magnetic disc, an optical recording medium such as an optical disc, a magneto-optical medium, and a computer-readable recording medium such as a semiconductor memory. In addition, it is possible to download the program via a communication network such as the Internet. Meanwhile, as a control device which performs such control, various types of devices other than a CPU can be applied, for example, a dedicated processor may be used.

The entire disclosure of Japanese Patent Application No. 2012-020766, filed Feb. 2, 2012, No. 2012-020767, filed Feb. 2, 2012 and No. 2012-219093 filed Oct. 1, 2012 are expressly incorporated reference herein.

What is claimed is:

1. A pulse wave measurement apparatus comprising:
a plurality of light receiving units that output measurement signals each indicative of a received amount of light which is irradiated to a measurement position where pulse waves are measured and passes through or is reflected from the measurement position;
an independent component analysis unit that performs an independent component analysis on the measurement signals which are output from the plurality of light receiving units, divides each of the measurement signals into a plurality of components, and calculates weighting factors of each of the plurality of components;
a dispersion calculation unit that calculates a value indicative of a degree of dispersion of values of the weighting factors which are calculated by the independent component analysis unit for each of the plurality of components;
a component specification unit that specifies a component of the plurality of components whose value indicative of the degree of dispersion calculated by the dispersion calculation unit satisfies a predetermined condition; and
a pulse wave information generation unit that generates pulse wave information indicative of the pulse waves using the component specified by the component specification unit.

2. The pulse wave measurement apparatus according to claim 1,
wherein the pulse wave information generation unit extracts the component specified by the component specification unit by performing an operation using the weighting factors calculated by the independent component analysis unit on the measurement signals which are output from the plurality of light receiving units, and generates the pulse wave information indicative of the pulse waves based on the extracted component.

3. The pulse wave measurement apparatus according to claim 2,
wherein the component specification unit specifies a component whose value indicative of the degree of dispersion calculated by the dispersion calculation unit is the smallest.

4. The pulse wave measurement apparatus according to claim 2,
wherein the component specification unit specifies a component whose value indicative of the degree of dispersion calculated by the dispersion calculation unit is within a predetermined range.

5. The pulse wave measurement apparatus according to claim 2,
wherein, when the plurality of components are specified by the component specification unit, the pulse wave information generation unit generates the plurality of specified components, and generates the pulse wave information based on the sum of the plurality of generated components.

6. The pulse wave measurement apparatus according to claim 1, further comprising:
a measurement signal specification unit that specifies a measurement signal whose value of the weighting factor of the component specified by the component specification unit satisfies the predetermined condition from among the measurement signals output from the plurality of light receiving units,
wherein the pulse wave information generation unit generates the pulse wave information indicative of the pulse waves based on the measurement signal specified by the measurement signal specification unit.

7. The pulse wave measurement apparatus according to claim 6,
wherein the component specification unit specifies a component whose value indicative of the degree of dispersion calculated by the dispersion calculation unit is greatest.

8. The pulse wave measurement apparatus according to claim 7,
wherein the measurement signal specification unit specifies a measurement signal whose value of the weighting factor of the component specified by the component specification unit is smallest.

9. The pulse wave measurement apparatus according to claim 6,
wherein the pulse wave information generation unit removes the component specified by the component specification unit from the measurement signal specified by the measurement signal specification unit, and generates the pulse wave information based on the measurement signal from which the component is removed.

10. A program causing a computer to perform:
receiving measurement signals from a plurality of light receiving units that output the measurement signals each indicative of a received amount of light which is irradiated to a measurement position where pulse waves are measured, and passes through or is reflected from the measurement positions;
performing an independent component analysis on the measurement signals which are output from the plurality of light receiving units, dividing each of the measurement signals into a plurality of components, and calculating weighting factors of each of the plurality of components;
calculating a dispersion value indicative of a degree of dispersion of values of the weighting factors which are calculated in the independent component analysis for each of the plurality of components;
specifying a component of the plurality of components whose value indicative of the degree of dispersion calculated in the calculating of the dispersion value satisfies a predetermined condition; and
generating pulse wave information indicative of the pulse waves using the component specified in the specifying of the component.
